(19) United States
(12) Patent Application Publication
(10) Pub. No.: US 2005/0042287 A1
(43) Pub. Date: Feb. 24, 2005

(54) Skin preparation composition

(76) Inventors: James G. Chaussee, Racine, WI (US); Debra A. Strasser, Franksville, WI (US)

Correspondence Address: S.C. JOHNSON & SON, INC., 1525 HOWE STREET, RACINE, WI, 53403-2236, US

(21) Appl. No.: 10/643,500

(22) Filed: Aug. 19, 2003

(51) Int. Cl. A61K 7/06; A61K 7/11

(52) U.S. Cl. 424/466

(57) ABSTRACT

According to one aspect of the present invention a composition comprises a intermediate and a diluent. About 2.8% to about 22.2% by weight of the intermediate comprises surfactant system and about 0.9% to about 3.9% by weight of the intermediate comprises a mixture of moisturizing compounds, wherein the mixture of moisturizing compounds includes at least 0.4% by weight of the total composition of lactic acid. About 4.2% to about 6.0% by weight of the diluent comprises a blend capable of producing CO₂ in situ.

### TECHNICAL FIELD

The present invention relates to skin preparation compositions for use in a variety of settings including use as a skin preparation for shaving. More particularly, it relates to skin preparation compounds containing lactic acid and utilizing a carbon dioxide propellant.

### BACKGROUND ART

A wide variety of skin preparation compositions and shaving preparations are known and available in the marketplace. For example, Monson, U.S. Pat. No. 5,902,225 discloses a post foamable multiple-sequential-foaming composition, utilizing carbon dioxide as a propellant produced from a bicarbonate/citric acid mix. A mix of surfactants and polymers including carbomer, decyl polyglucsoe, ammonium cocyl isethionate and others are utilized along with the propellant to produce a shaving cream.

Lentini et al., U.S. Pat. No. 6,177,092 discloses a self-foaming cleaning system that utilizes separate components in separate containers to produce a carbon dioxide propellant in a cleaning system. The propellant may include many different acids such as citric acid, lactic acid and ascorbic acid combined with sodium or potassium bicarbonate to produce the carbon dioxide.

Szymczak, U.S. Pat. No. 6,352,689 discloses a post-foaming shaving gel including poly(ethylene oxide) and polyvinylpyrrolidone in a preferred range of weight ratios. The shaving gels disclosed utilize triethanolamine LFG, vitamin E acetate, 50 percent panthenol, aloe vera and others in various combinations in the gels.

Skin preparations, and in particular shaving preparations, must achieve a multitude of factors including skin protection, humectancy, exfoliation, and the like. The balance of these factors improves the likelihood of the commercial success of the composition.

### SUMMARY OF THE INVENTION

According to one aspect of the present invention a composition comprises an intermediate and a diluent. About 2.8% to about 22.2% by weight of the intermediate comprises a surfactant and about 0.9% to about 3.9% by weight of the intermediate comprises a mixture of moisturizing compounds, wherein the mixture of moisturizing compounds includes at least 0.4% by weight of the total composition of lactic acid. About 4.2% to about 6.0% by weight of the diluent comprises a blend capable of producing CO₂ in situ.

According to another aspect of the present invention skin preparation that is a shaving cream having an intermediate and a diluent comprises about 0.4% to about 2% by weight of the intermediate comprises a lactic acid; and about 4.2% to about 6.0% by weight of the diluent comprises a blend capable of producing CO₂.

Other aspects and advantages of the present invention will become apparent upon consideration of the following detailed description.

### BRIEF DESCRIPTION OF THE DRAWINGS

FIGS. 1-4 are photographs of the formulation of U.S. Pat. No. 5,902,225 10, 20, 30, and 60 seconds after being dispensed at 85 psig;

FIGS. 5-8 are photographs of the formulation of U.S. Pat. No. 5,902,225 10, 20, 30, and 60 seconds after being dispensed at 40 psig;

FIGS. 9-12 are photographs of a formulation of current invention 10, 20, 30, and 60 seconds after being dispensed at 85 psig;

FIGS. 13-16 are photographs of a formulation of current invention 10, 20, 30, and 60 seconds after being dispensed at 40 psig.

### DETAILED DESCRIPTION OF THE PREFERRED EMBODIMENTS

The present invention is directed to an improvement in skin preparation compositions. The composition includes an intermediate including a surfactant and a mixture of moisturizers including lactic acid, and a diluent including a blend capable of producing CO₂ in situ as described in further detail below. The formulation may also include polymers, preservatives, colorants, other surfactants, fragrances, vitamins, emollients, pigments, foam stabilizers and humectants.

The surfactant of the present invention may be any surfactant known to one of skill in the art. Additionally, a mix of a variety of surfactants can also be employed such as nonionic and anionic surfactants in combination. In the preferred embodiment the surfactant includes three different surfactants to fill a variety of roles. Preferably, the surfactant comprises about 2.8% to about 22.2% by weight of the intermediate; and most preferably the surfactant comprises about 12% by weight. Decyl polyglucose, commercially available as Plantaren 2000N from Congis Corporation, of Cincinnati, OH is a nonionic surfactant that serves as a roamer, cleanser, and lather stabilizer in the intermediate. Preferably, the decyl polyglucose comprises about 0.1% to about 16% by weight of the intermediate; and most preferably comprises about 8.0% by weight. Ammonium cocyl isethionate, commercially available as Jordapon ACI-30 from BASF Corporation of Parsippany, N.J. is an anionic surfactant that serves as a roamer, wetting agent, and afterfeel enhancer in the intermediate. Preferably, the ammonium cocyl isethionate comprises about 2.4% to about 4.2% by weight of the intermediate; and most preferably comprises about 3.3% by weight. Sodium dioctyl sulfosuccinate, commercially available as Aerosol OT-70 PG from Cytec Industries, Inc. of West Patterson, N.J. is another anionic surfactant that serves as a flash foamer and wetting agent in the intermediate. Preferably, the sodium dioctyl sulfosuccinate comprises about 0.3% to about 2% by weight of the intermediate; and most preferably comprises about 0.5% by weight. While the above listed surfactants are preferable other surfactants known to one of skill in the art may be utilized within the scope of the present invention.

The mixture of moisturizers includes lactic acid and preferably includes a combination of moisturizing compounds. The included moisturizers can fill a variety of roles within the intermediate including but not limited to a moisturizer role, a humectant and/or an exfoliant. Preferably, the mixture of moisturizers comprises about 0.9% to about 3.9% by weight of the intermediate; and most preferably comprises about 1.6% by weight. The lactic acid is preferably, but not necessarily, of a buffered nature for ease of handling the lactic acid prior to inclusion in the intermediate. The buffered lactic acid, commercially available as Purac BF P-41 from PURAC America of Lincolnshire, Ill. is an organic acid which serves as a humectant and exfoliant. Preferably, the lactic acid comprises about 0.4% to about 2.5% by weight of the intermediate; and most preferably comprises about 0.5% by weight. In the preferred embodiment of the mixture of moisturizers vitamin E acetate is utilized to serve as a moisturizer. Preferably, the vitamin E acetate comprises about 0.1% to about 0.2% by weight of the intermediate; and most preferably about 0.1% by weight. Pro-vitamin B-5 is also included in the preferred embodiment, commercially available as Liquid dl Panthenol 50% CG from Roche, Parsippany, N.J. and serves as a moisturizer and humectant. Preferably, the pro-vitamin B-5 comprises about 0.5% to about 1.0% by weight of the intermediate; and most preferably comprises about 1.0% by weight. Also included in the preferred embodiment is aloe vera gel 200%, commercially available as Covera Dry from various producers that serves as an additional moisturizer. Preferably, the aloe vera gel 200% comprises about 0.01% to about 0.05% by weight of the intermediate; and most preferably comprises about 0.03% by weight. Also preferably, the weight ratio of lactic acid to other moisturizer is in the range of about 1:2 to about 1:2.5. While the preferred embodiment described above includes specific compounds in combination with lactic acid to comprise the mixture of moisturizers, one of skill in the art would recognize that other compounds could be used within the scope of the present invention.

The intermediate, in this preferred embodiment, also includes a blend of polymers. Preferably, the blend of polymers comprises about 0.8% to about 1.1% by weight of the intermediate; and most preferably about 0.95% by weight. In a preferred embodiment the blend of polymers includes a carbomer, a hydroxyethylcellulose, and a hydroxypropylmethylcellulose. The carbomer, commercially available as Carbopol ETD 2020 from Noveon, Inc. of Cleveland, Ohio is a polyacrylic acid polymer that serves as a thickener. Preferably, the carbomer comprises about 0.5% to about 0.8% by weight of the intermediate; and most preferably about 0.65% by weight. The hydroxyethylcellulose, commercially available as Natrocel K4MS from Hercules, Inc of Wilmington, Del. is a cellulose polymer that serves as a thickener and lubricant. Preferably, the hydroxyethylcellulose comprises about 0.15% to about 0.25% by weight of the intermediate; and most preferably about 0.2% by weight. The hydroxypropylmethylcellulose, commercially available as Methocel K4MS from Colorcon of West Point, Pa. is another cellulose polymer and serves as a thickener, lubricant and lather stabilizer. Preferably, the hydroxypropylmethylcellulose comprises about 0.08% to about 0.12% by weight of the intermediate; and most preferably about 0.1% by weight. However, while certain polymers are listed in this preferred embodiment one of skill in the art would know to substitute similar polymers and still be within the scope of the present invention.

A most preferred embodiment of a skin preparation composition further includes other compounds and chemicals which play a variety of roles with in the final composition. For example, the intermediate may include 99% Triethanolamine LFG (50%) preferably comprising about 0.6% to about 0.8% by weight; and most preferably about 0.67% by weight. The triethanolamine serves to balance the pH of the composition and to assist in polymer blend by uncurling the carbomer. Furthermore, the intermediate may include various preservatives, fragrances and the like that increase composition shelf life and consumer desirability. The remainder of the intermediate is then preferably deionized water.

In the preferred embodiment the composition includes a diluent including a blend capable of producing CO₂ including potassium bicarbonate and citric acid in about a 1:1 mole relationship. This equates to preferably about 45% to about 47% by weight of the diluent comprising potassium bicarbonate and about 13% to about 15% by weight of the diluent comprising citric acid; and most preferably about 46.7% potassium bicarbonate and about 14.2% citric acid. In addition, the diluent may include a post foaming agent to assist in lathering the composition, preferably a hydrocarbon blend such as a 3/97 blend of isobutane/isopentane; however, other post-foaming agents could be used. The diluent and intermediate are then preferably combined in a ratio of intermediate to diluent in the range of about 88:12 to about 92:8 and most preferably combined at the ratio of about 90.3:9.7. When combined in the composition and stored in a preferred container produces about 85 psig in the container before any product has been dispensed. As discussed below, this pressure creates the optimum bubble consistency as a starting pressure and for the dispensing life of the composition. While a citric acid and potassium bicarbonate blend is utilized in this preferred embodiment, other blends capable of producing CO₂ are known to those of skill in the art and may be utilized within the scope of this invention.

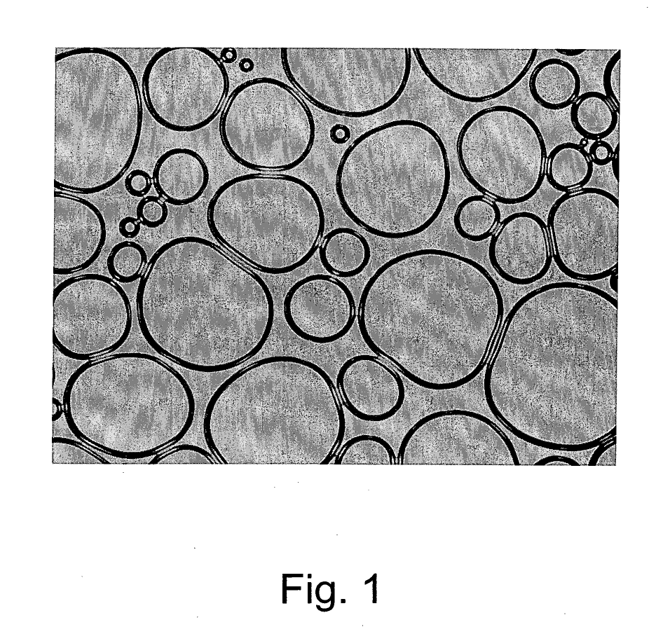

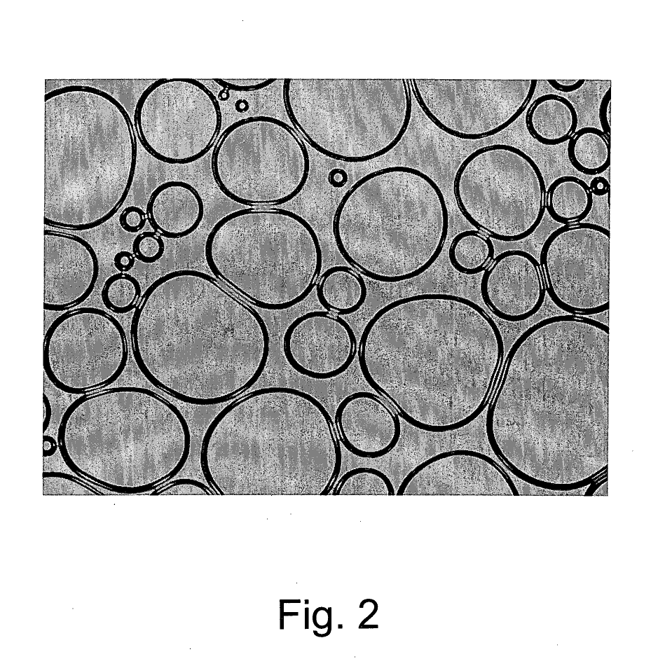

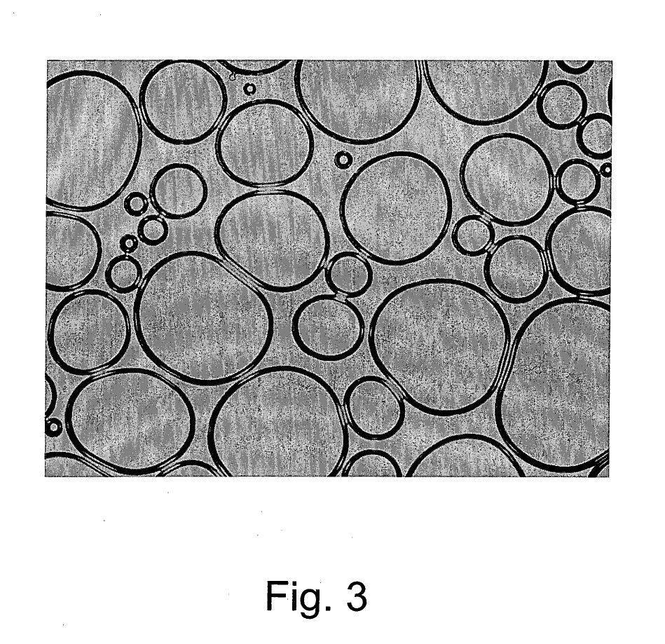

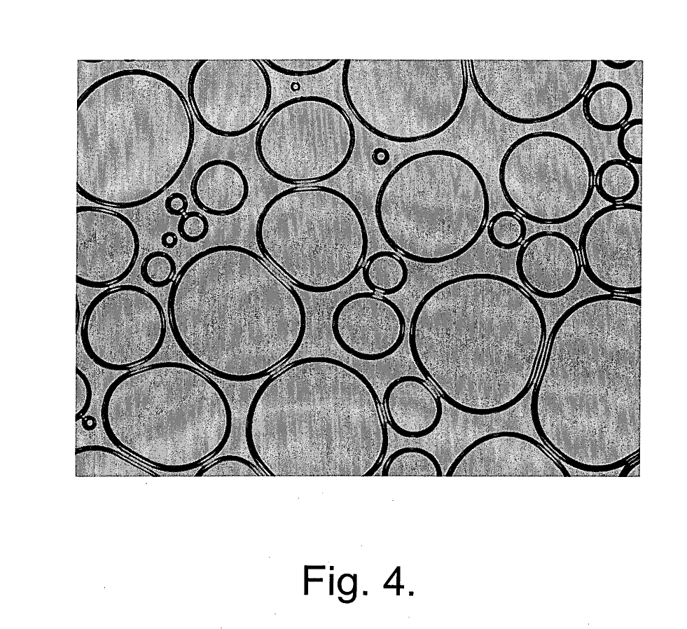

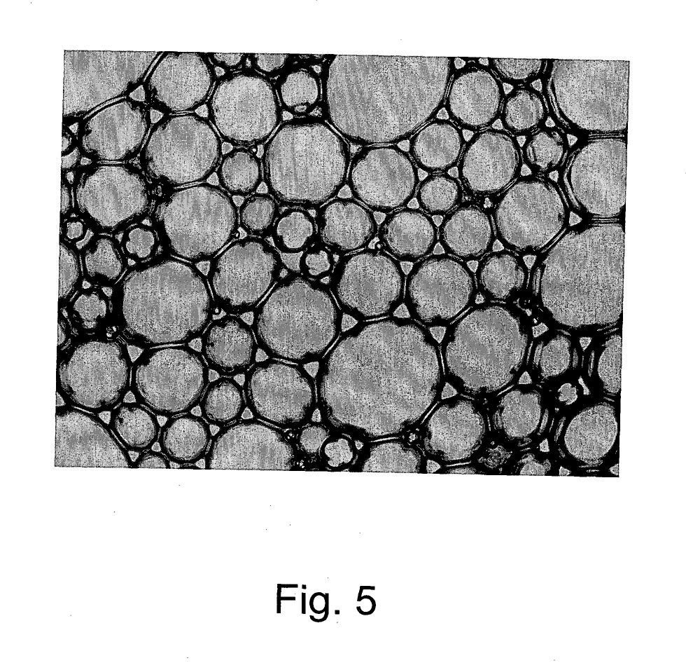

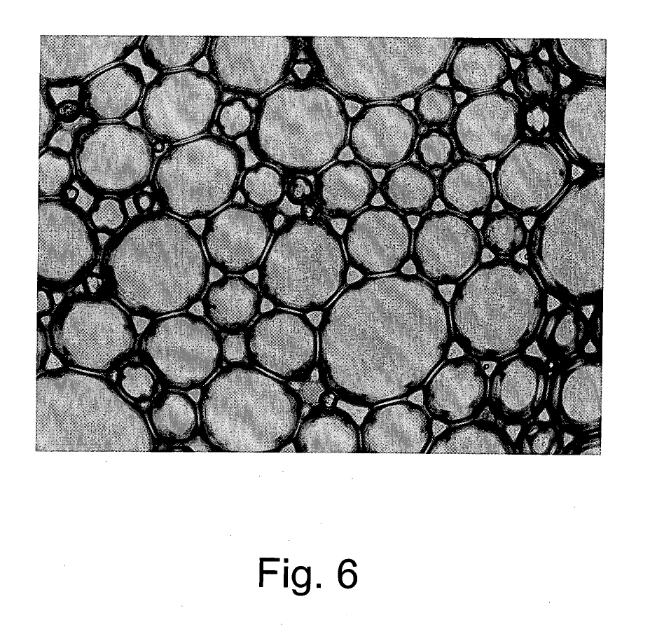

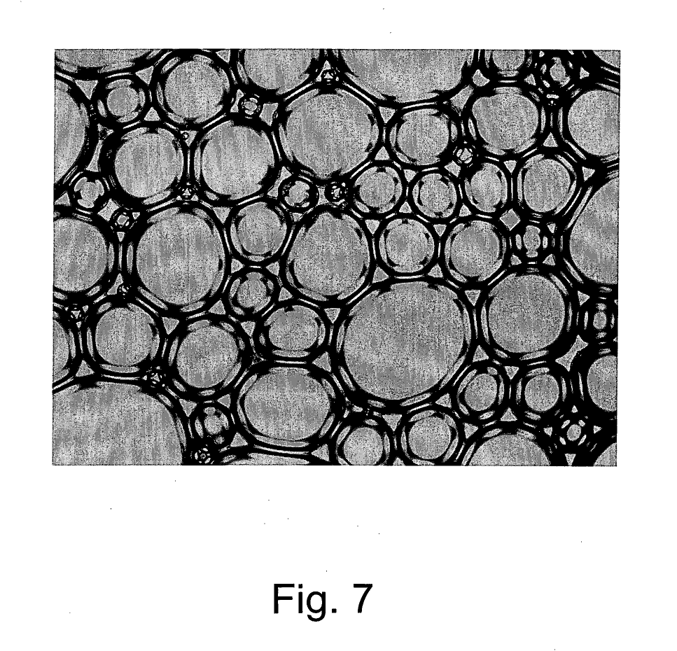

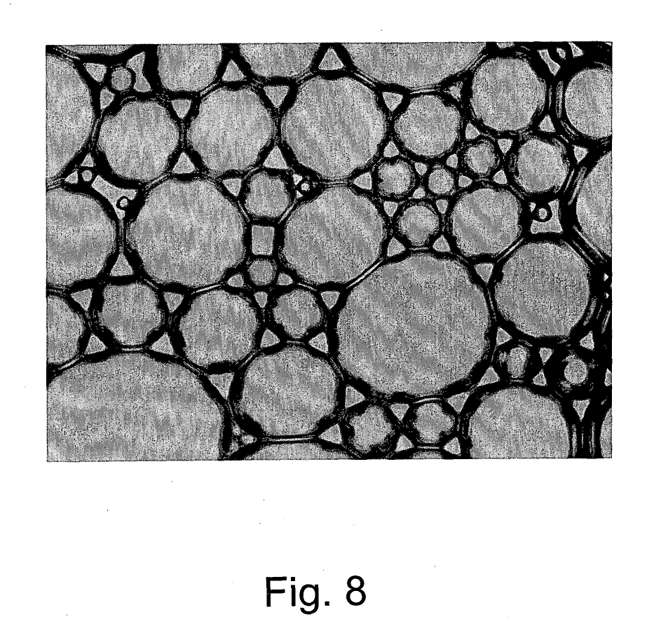

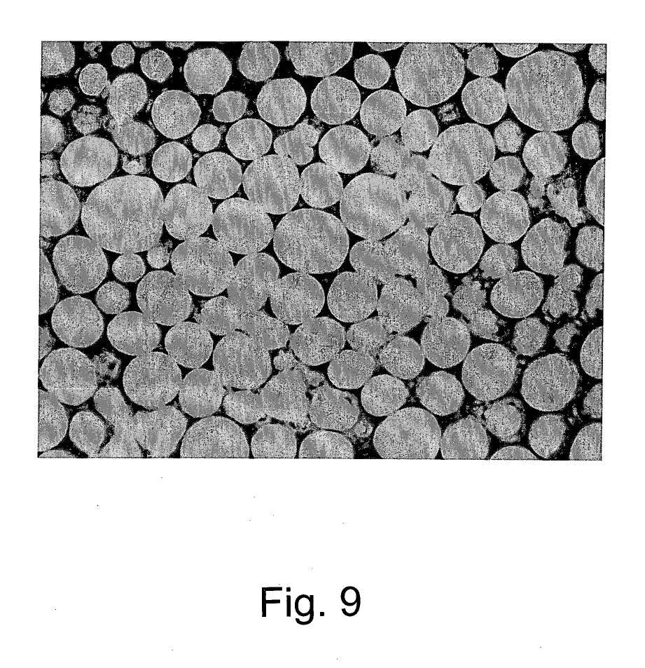

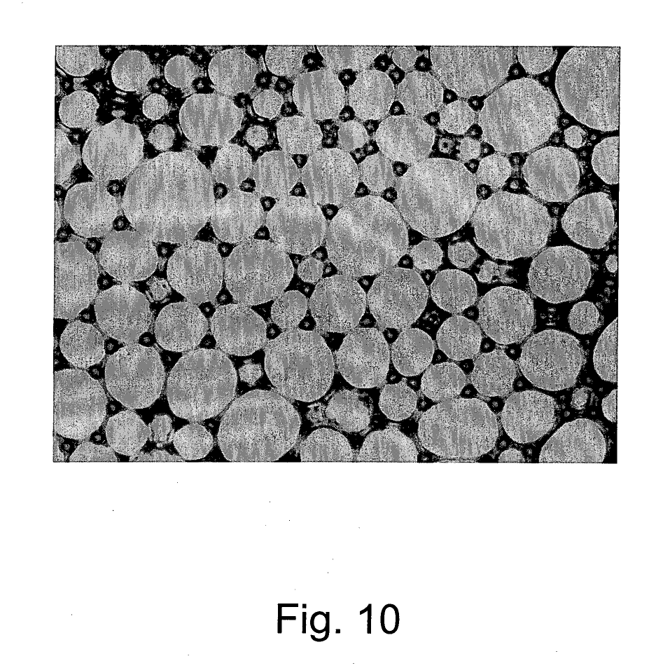

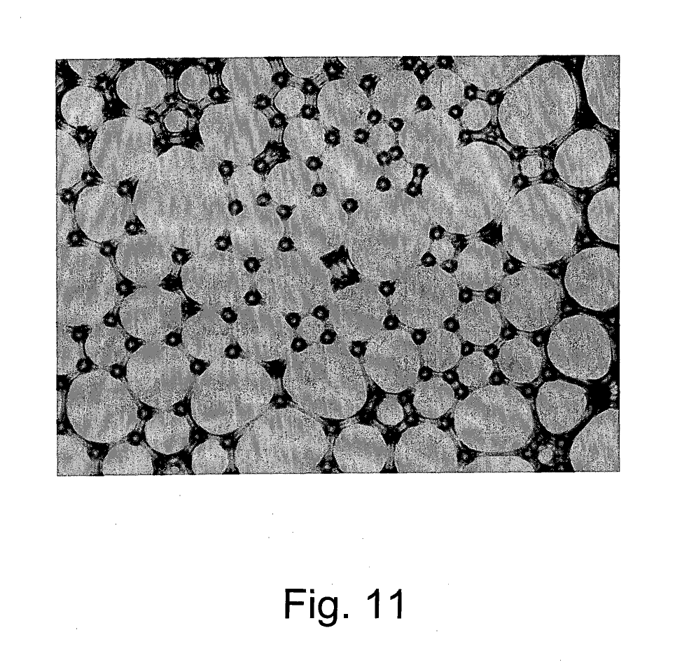

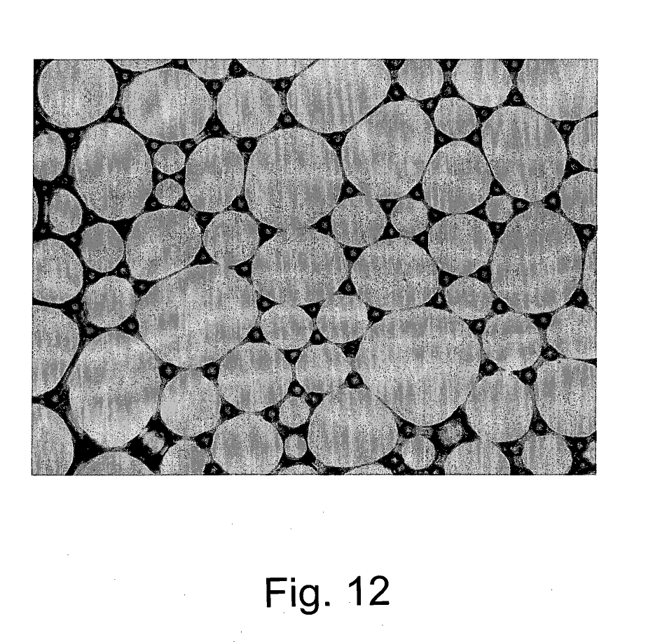

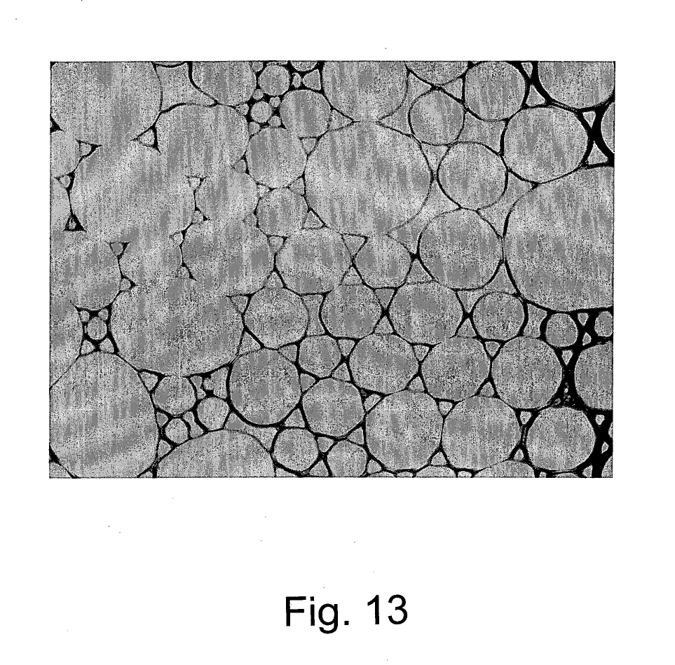

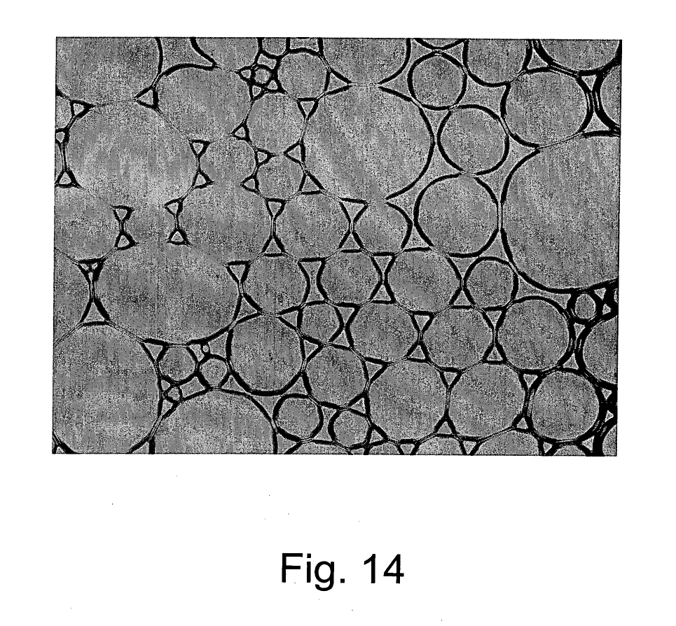

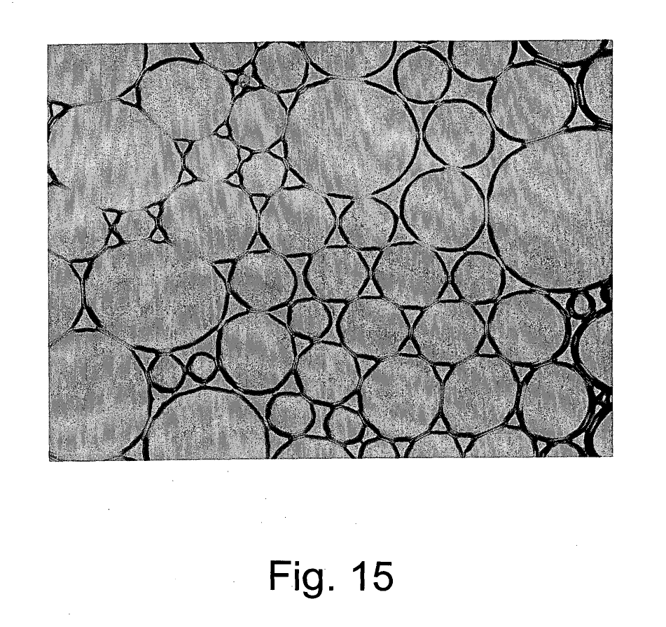

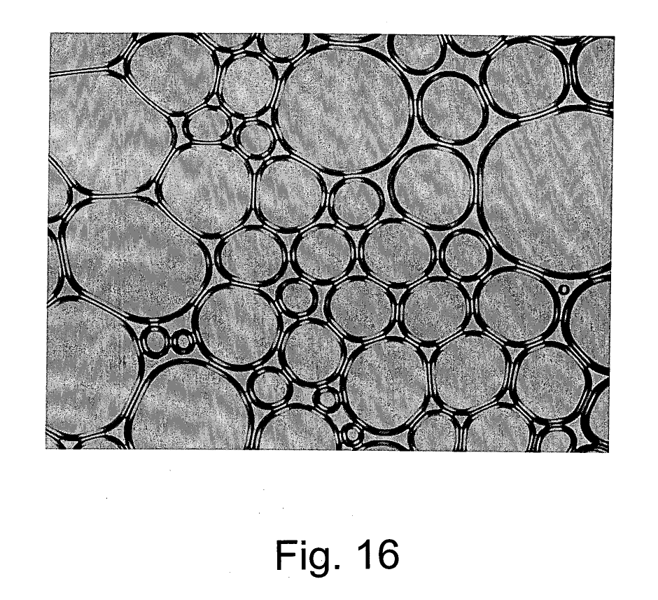

Preferably, ratio of intermediate to diluent is such that enough CO₂ is produced so that when about 90% of the composition has been dispensed the composition is still under at least 40 psig. In a skin preparation composition, especially in one used as a preparation for shaving, the size and numbers of bubbles produced are important factors for use as a lubricant and humectant. The smaller the bubbles, the more surface area is present to hold water to the skin of a user and thus easier it is shave. As can be seen in FIGS. 1-16 the pressure of dispensing can impact the size and number of bubbles. FIGS. 1-4 show a prior art composition according to Monson U.S. Pat. No. 5,902,225 dispensed at a pressure of 85 psig. FIGS. 5-8 show the same composition dispensed at a pressure of 40 psig. Comparatively, FIGS. 9-12 show the formulation shown in Example 1 below dispensed at 85 psig and FIGS. 13-16 show the same formulation dispensed at 40 psig. Each series of photos shows a time span of 10 seconds, 20 seconds, 30 seconds and 60 seconds after dispensing with dispensing from identical dispensing apparatus. As can be seen, a composition according to the present invention dispensed at 85 psig results in a greatly increased number of bubbles and a smaller bubble size both throughout the exposure time. However, even at the lesser pressure of 40 psig the current formulation results in a smaller bubble size throughout the time span.

### EXAMPLE

The following is an example of a formulation according to the present invention. All the percentages are weight percentages.

| Constituent | % |
|---|---|
| Intermediate: | |
| Carbopol ETD 2020 | 0.65 |
| Pantaren 2000 N | 8.00 |
| Jordapon ACI 30 | 3.30 |
| Lactic Acid | 0.50 |
| m Paraben | 0.30 |
| p Paraben | 0.10 |
| Fragrance | 0.40 |
| 99% TEA LFG (50%) | 0.67 |
| Vitamin E Acetate | 0.10 |
| Panthenol Liquid 50% | 1.00 |
| Aloe Vera 200% | 0.03 |
| Methocel K4MS | 0.10 |
| Natrosol 250 HHR | 0.20 |
| Aerosol OT-70 PG | 0.50 |
| Water | 84.15 |
| Diluent: | |
| Potassium Bicarbonate | 46.7 |
| Citric Acid | 14.2 |
| Isobutane | 9.2 |
| Isopentane | 29.9 |
| Dilution Ratio: | |
| Intermediate | 90.3 |
| Diluent | 9.7 |

Industrial Applicability

The present invention provides compositions useful as a skin preparation in general and more specifically to skin preparations to be used in shaving preparation. The present invention provides a composition that is generally a flowable shaving cream type product rather than a gel as generally used in the art and is particularly suited to shaving preparation for legs and other body parts other than the face, although such a use is possible. The preferred embodiment provides increased moisturizing with better and more consistent lathering properties.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

1. A composition, comprising:
an intermediate;
a diluent;
about 2.8% to about 22.2% by weight of the intermediate of a surfactant;
about 0.9% to about 3.9% by weight of the intermediate a mixture of moisturizing compounds, wherein the mixture of moisturizing compounds includes at least 0.4% by weight of the intermediate of lactic acid;
about 58% to about 62% by weight of the diluent being a blend capable of producing CO₂ in situ.

2. The composition of claim 1, wherein the mixture of moisturizing compounds includes lactic acid and an additional moisturizing compound selected from a group consisting of aloe vera gel 200%, Pro-Vitamin B5, Vitamin E Acetate and mixtures thereof.

3. The composition of claim 2, wherein the weight ratio of lactic acid to additional moisturizer is in the range of about 1:2 to about 1:2.5.

4. The composition of claim 1, wherein the intermediate further includes about 0.8% to 1.1% by weight a blend of polymers.

5. The composition of claim 4, wherein the blend of polymers is selected from the group consisting of carbomer, hydroxypropylmethylcellulose, hydroxyethycellulose and mixtures thereof.

6. The composition of claim 1, wherein the diluent further includes a hydrocarbon propellant for use as a post-foaming agent.

7. The composition of claim 6, wherein the hydrocarbon propellant is an isobutane/isopentance blend.

8. The composition of claim 7 wherein the weight ratio of isobutane to isopentane is in the range of about 3:97.

9. The composition of claim 1, wherein the lactic acid is in the form of a buffered lactic acid.

10. The composition of claim 1, wherein the surfactant includes at least one from the group consisting of decyl polyglucose, ammonium cocyl isethionate, sodium dioctyl sulfosuccinate, and mixtures thereof.

11. The composition of claim 10, wherein the surfactant system includes decyl polyglucose, ammonium cocyl isethionate, and sodium dioctyl sulfosuccinate.

12. The composition of claiml, wherein the blend capable of producing CO₂ comprises a blend of potassium bicarbonate and citric acid.

13. The composition of claim 12, where the weight ratio of potassium bicarbonate to citric acid is about 3.0:1.3.

14. The composition of claim 1, further including about 0.6% to about 0.8% by weight of the intermediate a triethanolamine.

15. The composition of claim 1, wherein the composition is stored in a container at an initial pressure of about 85 psig.

16. The composition of claim 1, wherein the composition is dispensed from a container at a pressure of at least 40 psig.

17. A shaving cream having an intermediate and a diluent, comprising:
about 0.4% to about 2% by weight of the intermediate a lactic acid;
about 58% to about 62% by weight of the diluent a blend capable of producing CO₂.

18. The shaving cream of claim 17, further including about 2.8% to about 22.2% by weight of the intermediate a surfactant system.

19. The shaving cream of claim 18, further including about 0.5% to about 1.4% by weight of the intermediate a moisturizer selected from a group consisting of aloe vera gel 200%, Pro-Vitamin B5, Vitamin E Acetate and mixtures thereof.

20. The shaving cream of claim 17, wherein the composition is stored in a container at an initial pressure of about 85 psig.

21. The shaving cream of claim 17, wherein the composition is dispensed from a container at a pressure of at least 40 psig.
